(19) United States
(12) Patent Application Publication
(10) Pub. No.: US 2024/0166685 A1
(43) Pub. Date: May 23, 2024

(54) SYSTEMS AND METHODS FOR PROCESS SCALE ISOLATION OF A PROTEIN

(71) Applicant: Plasma Technologies, LLC, Charleston, SC (US)

(72) Inventors: Eugene Zurlo, Charleston, SC (US); Dennis Curtin, Charleston, SC (US); Klaus Peter Radtke, Apex, NC (US); Ryan Dorfman, Essex, VT (US); Matthew Whelihan, Colchester, VT (US)

(21) Appl. No.: 18/427,623

(22) Filed: Jan. 30, 2024

Related U.S. Application Data
(63) Continuation-in-part of application No. 18/516,062, filed on Nov. 21, 2023.
Application No. 18/516,062 is a division of application No. 17/560,163, filed on Dec. 22, 2021, now Pat. No. 11,884,702.

(51) Int. Cl. C07K 1/30; C07K 1/22; C07K 1/36; C07K 16/06

(52) U.S. Cl. CPC C07K 1/303; C07K 1/22; C07K 1/36; C07K 16/065

(57) ABSTRACT

Systems and methods are described in which proteins are isolated from complex solutions in high yield and at high purity. Such systems and methods are carried out at ambient temperature and can be carried out at industrial scale with minimal energy requirements and minimal carbon footprint, using successive chromatographic separations that retain the protein or proteins of interest in flow-through fractions. At least one of the chromatography media used is selected to be capable of interacting with both contaminants and the protein of interest, however capacity of this media is selected such that the protein of interest is displaced and remains in the flow-through. Methods for isolation of IgG, albumin, and both IgG and albumin are provided.

This application is a continuation in part of U.S. patent application Ser. No. 18/516,062, filed Nov. 21, 2023; which claims the benefit of U.S. patent application Ser. No. 17/560,163, filed Jan. 6, 2022; which claims the benefit of United States Provisional Patent Application Nos. 63/131,097 filed on Dec. 28, 2020, U.S. Provisional Patent Application No. 63/208,778, filed Jun. 9, 2021; and U.S. Provisional Patent Application No. 63/544,576, filed on Oct. 17, 2023. This application also claims the benefit of U.S. patent application Ser. No. 17/560,219, filed on Dec. 22, 2021; which claims the benefit of U.S. Provisional Patent Application No. 63/272,605, filed Oct. 17, 2021. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is the isolation of proteins from heterogeneous protein solutions.

### BACKGROUND

The background description includes information that may be useful in understanding the present invention. It is not an admission that any of the information provided herein is prior art or relevant to the presently claimed invention, or that any publication specifically or implicitly referenced is prior art.

Isolation of proteins at high purity generally requires the use of multiple purification steps, at least one of which is a chromatography step. Chromatography can be performed in either positive selection mode (in which the protein of interest binds to and is later eluted from the chromatography media following a washing step) or negative selection mode (in which the protein of interest appears in a flow-through fraction).

Chromatography that utilizes positive selection is generally used to provide proteins of high purity (e.g., exceeding 80% purity by weight). For example, affinity chromatography media is typically used to bind a protein of interest from a sample that includes contaminants to the affinity media. Following washing steps to remove contaminants, the protein of interest is then eluted from the affinity media in an elution buffer at high purity, for example by applying a low pH buffer. Unfortunately, such elution is generally incomplete, and the elution buffer used can result in denaturation of the protein being isolated.

Methods for isolation of a protein at high purity typically utilize multiple chromatography steps. For example, in a typical isolation process for immunoglobulin G (IgG) an anion exchange chromatography media is used in negative selection mode, with IgG appearing in the flow-through and some contaminants binding to the anion exchange media. The anion exchange media is used in conjunction with a cation exchange chromatography media that is used in positive selection mode, which binds the IgG and allows remaining contaminants pass in the flow-through. Following a wash step, the bound IgG is eluted at high purity using a buffer with high ionic strength. This elution step, however, is generally incomplete.

In using such a conventional purification strategy for a therapeutic protein product, the loss of target protein is particularly consequential when applied to a relatively pure solution with low concentrations of contaminants (which nevertheless need to be removed due to their adverse side effects at even femtomolar concentrations). For example, low concentrations of coagulation proteins, or host cell proteins often need to be removed due to their potential for severe adverse events.

Unfortunately, binding to and subsequent elution from chromatography media necessarily involves the use of large volumes of chromatography media. Such large-scale binding and elution methods also require significant time in order to provide for adequate loading, washing, and elution of the large volumes of chromatography media required. In addition, a chromatography medium with mechanical properties that make it suitable for implementation in large volume (e.g., greater than 5 L) columns may not have suitable binding characteristics. In addition, chromatography methods that include binding and elution steps inevitably result in significant loss (e.g., up to 10% or more) of the target protein, which can be exacerbated by prolonged occupancy on a large volume chromatography column. While processes that utilize positive selection are useful on a small scale, application, process complexity, time requirements, and material limitations (in terms of both costs and physical limits of the chromatography media) render them unsuitable for large scale protein isolation processes (which can involve processing of 8,000 liters or more of starting material).

Thus, there is still a need for rapid, efficient, and scalable methods for isolation of proteins at high yield and purity.

### SUMMARY OF THE INVENTION

The inventive subject matter provides systems and methods in which proteins are isolated from a complex solution using one or more chromatographic separations that retain the protein of interest in the flow-through. At least one of the chromatography media used is selected to be capable of interacting with both contaminants and the protein of interest, however capacity of this media is selected such that the protein of interest is displaced and remains in the flow-through.

One embodiment of the inventive concept is a method for reducing an environmental impact of industrial scale isolation of a first protein from a blood product (such as a blood plasma or a product of processing blood plasma) by adding a precipitating salt to a volume of the blood product comprising the first protein, a second protein, a third protein, and a fourth protein (e.g., at ambient or an elevated temperature) to generate a supernatant and a precipitate, where the blood product has a volume of at least 2,000 L (e.g. 2,000 L to 5,000 L or more) or is derived from at least 2,000 L (e.g., 2,000 L to 5,0000 L or more) of blood plasma.

Subsequently one of the supernatants or one of the precipitates is applied to a first ion exchange media with a first charged group to generate a first flow-through fraction that includes the first protein and the third protein. In some embodiments the supernatant or the precipitate can be passed through a filter prior to application to the first chromatography media. Such a filter can be selected to selectively bind an activated clotting factor. The second protein is retained on the first chromatography media. This first flow-through fraction is applied to a second ion exchange media with a second charged group to generate a second flow-through fraction that contains the first protein. The first charged group and the second charged group have opposing charges, and the chromatography conditions are selected such that both the first protein and the third protein can bind to the second ion exchange media. In such methods steps (1) to (3) are performed at ambient temperature and are completed in less than 24 hours from initiation of step (1). In some embodiments the blood product is a product of a salt precipitation step applied to a blood plasma. In some embodiments the redissolved precipitate is applied to the first chromatography media and the first protein is IgG. In such methods the environmental impact can be energy consumption, and when the blood product is cryo-poor plasma, the first protein is IgG, and the precipitating salt is a citrate salt. Such a method can provide an 89% reduction in energy requirements relative to prior art ethanol-precipitation methods for isolating IgG from cryo-poor plasma at the volume. Methods of the inventive concept the method can not include steps for recovering or reclaiming an organic solvent (such as ethanol), however they can include steps for capturing and/or recycling the precipitating salt.

In some embodiments of the inventive concept the fourth protein can be recovered from the supernatant. In such embodiments the fourth protein can be albumin. Such methods can include a step of adding caprylate to the supernatant to generate a caprylate treated supernatant and raising the temperature of the caprylate treated supernatant to about 60° C. to generate a third supernatant and a third precipitate, where the third supernatant includes a crude albumin. Some embodiments include a step of adjusting pH of the third supernatant to from pH 4.5 to pH 6.5 and applying it to an anion exchange media to generate a third flow-through fraction containing a purified albumin. Some embodiments include a further step of applying this third flow through to a cation exchange media to generate a fourth flow-through that includes a polished albumin. In such embodiments capacity of this cation exchange media can be selected to bind less than 30% of albumin content of the third flow-through.

Various objects, features, aspects and advantages of the inventive subject matter will become more apparent from the following detailed description of preferred embodiments.

### BRIEF DESCRIPTION OF THE DRAWINGS

FIG. 1: FIG. 1 schematically depicts a prior art method for isolation of proteins.

FIGS. 2A to 2C: FIGS. 2A and 2B schematically depict early and later stages of an exemplary method of the inventive concept, respectively. FIG. 2A schematically depicts precipitation steps of an exemplary method of the inventive concept. FIG. 2B schematically depicts chromatography steps of an exemplary method of the inventive concept. FIG. 2C schematically depicts an exemplary method for recovery of a precipitating salt utilized in a method of the inventive concept.

FIG. 3: FIG. 3 schematically depicts an exemplary process of the inventive concept.

FIG. 4: FIG. 4 schematically depicts an alternative exemplary process of the inventive concept.

FIG. 5: FIG. 5 schematically depicts an exemplary process of the inventive concept as applied to isolation of immunoglobulin G (IgG) from a blood product.

FIG. 6: FIG. 6 provides a histogram displaying annual energy requirements of steps of prior art methods and methods of the inventive concept..

FIG. 7: FIG. 7 provides a photograph of an exemplary electrophoresis study of caprylate precipitation in a step of a method of the inventive concept.

FIG. 8: FIG. 8 schematically depicts a method of the inventive concept for isolation of albumin using flow-through chromatographic steps.

FIG. 9: FIG. 9 schematically depicts a method of the inventive concept that provides an exemplary combined method of the inventive concept for isolation of both IgG and albumin using flow-through chromatographic steps.

### DETAILED DESCRIPTION

The Inventors have developed a simple and scalable process that can isolate a protein of interest at high yield (e.g., greater than 60% of protein content of the starting material) and high purity (e.g., 70%, 75%, 80%, 85%, 90%, 95%, 98%, 99%, by weight or greater). These methods are particularly useful in isolation of a protein of interest from a solution that includes one or more contaminants that have chromatographic behavior that is similar to that of the protein of interest (e.g., binding to the same chromatography media). Surprisingly, at industrial scale these processes were found to provide a substantially reduced environmental impact in regard to energy consumption and generation of organic waste products when compared to prior art methodologies applied at the same scale. In addition, even at industrial scale such processes were able to be completed in substantially less time than prior art processes applied at the same scale.

Methods of the inventive concept utilize a chromatography media that is selected to bind both the protein that is to be isolated (i.e., the protein of interest) and one or more contaminants that are present in solution with the protein of interest. Such contaminants can include proteins or other molecules (e.g. lipids) present in the starting material that are not the protein of interest, and in some embodiments can be compounds that are added during pre-chromatography processing steps. The chromatography media can be selected to interact more strongly with one or more of the contaminants than the protein of interest. The capacity of the chromatography media used is calculated on the basis of the contaminant content of the solution containing the protein of interest, and is selected such that breakthrough of one or more contaminants does not occur to a significant extend (e.g., greater than 0.01%, 0.1%, 0.25%, 0.5%, 1%, 2%, 5%, or 10%) of the contaminant content of the solution being purified. It should be appreciated that such chromatography media capacity is a function of the nature of the chromatography media, the amount of media used, solution composition, solution pH, flow rate, etc., and is readily determined and/or optimized experimentally. Generally the capacity of chromatography media required is relatively small relative to prior art methods, and can advantageously be provided by a functionalized (e.g., charged) filter.

Within the context of this application the term “about” in association with a numeric value is understood to indicate a range that is within 10% of the named value. For example, the phrase “about 1” should be understood to indicate a range of 0.9 to 1.1 for the named value.

In practice Inventors have surprisingly found that, despite using chromatography media having the ability to capture the protein of interest under the buffer conditions used, application of methods of the inventive concept permit recovery of at least 70%, 75%, 80%, 85%, 90%, 95%, 97%, or 99% of the total protein of interest that is applied to chromatography media within the flow-through fraction recovered from the chromatography media.

Without wishing to be bound by theory, Inventors believe that interaction of contaminants with the chromatography media effectively displaces the protein of interest from the chromatography media. This permits highly efficient recovery of a protein of interest from contaminants having similar charge or other characteristics that would otherwise make them difficult to separate from one another. Purity of the recovered protein of interest can be from 80%. 85%, 90%, 95%, 98%, 99%, or higher (by weight).

In some embodiments of the inventive concept a starting or raw material can be subjected to one or more processing steps prior to application to chromatography media. Such processing steps include, but are not limited to, cryoprecipitation, selective precipitation (e.g., using a salt, hydrophilic polymer, and/or organic solvent), viral inactivation, lipid solvation and/or removal, etc. In preferred embodiments precipitation steps are performed at ambient temperature (i.e., non-refrigerated room temperature) that segregate proteins and other materials (e.g., lipids) into either supernatant or precipitate phases in order to remove or reduce contaminants. Performance at ambient temperatures greatly reduces processing time, energy consumption, and carbon footprint of such methods.

Suitable precipitation reactions can be performed by the addition of a salt (e.g., a citrate or acetate salt, a sulfate salt, a phosphate salt, etc.) or a hydrophilic polymer (e.g., polyethylene glycol, dextran, etc.) to a starting or raw material. For example, a precipitation step can be performed by adding sodium citrate to a starting or raw material (e.g., serum, plasma, cryo-poor plasma, cell culture products, etc.) to give a final concentration of from 1% w/v to 50% w/v. 3% w/v to 40% w/v, 5% w/v to 35% w/v, 10% w/v to 30% w/v, 12% w/v to 25% w/v, 15% w/v to 20% w/v. 15% w/v to 50% w/v, 20% w/v to 35% w/v, or any concentration range within these.

Such a salt can be added as a concentrated stock solution (e.g. 50% w/v or higher) in a volume sufficient to provide the desired final concentration. Alternatively, such a salt can be added directly as a dry or crystalline solid. Addition of a salt as a dry or crystalline solid can include the use of extensive mixing during addition, for example the use of a jet mixer. Similarly, addition of a salt as a dry or crystalline solid can include the use of a device to broadcast the salt over a significant portion (e.g., 25% or more) of the surface of the liquid into which it is being dispersed. Inventors have found that addition of a precipitating salt directly as a dry or crystalline solid advantageously reduces processing time, and have surprisingly found that the resulting precipitation products and distribution of proteins between them is not distinguishable from those generated by traditional gradual addition as a concentrated solution. For example, Inventors have found that addition of a citrate salt in dry or crystalline form to 5,000 L of cryo-poor plasma to provide a concentration sufficient for initial steps of IgG isolation (e.g., about 11% w/v) can be completed in about 30 minutes (e.g., using a jet mixer), whereas addition as stock solution (e.g., a 50% w/v solution) requires approximately 2.5 hours (e.g., as a flow-optimized surface spray).

Such salts or hydrophilic polymers can be provided at ambient temperature and added to a starting or raw material that is at ambient temperature, as their dissolution does not generate heat. As such, refrigeration of the starting material and/or the reagent used for precipitation is not required. In some embodiments a resulting supernatant, should it include the protein to be isolated, can be subjected to chromatography as described above. Alternatively, if the protein of interest is segregated into a precipitate so produced it can be suspended in water or a suitable buffer and dissolved prior to application to chromatography steps.

In some embodiments a supernatant that includes the protein to be isolated can be subjected to further precipitation steps to produce a second supernatant and a second precipitate in order to further remove or reduce contaminants. This can be accomplished, for example, by addition of additional salt or hydrophilic polymer to the supernatant. Alternatively, a precipitate can be suspended and dissolved in water or a suitable aqueous buffer and similarly subjected to a second precipitation step to produce a second supernatant and second precipitate. Such a second precipitate can be suspended in water or a suitable aqueous buffer and dissolved prior to application to chromatography media. In some embodiments a supernatant or a dissolved precipitate can be subjected to buffer exchange (e.g., by diafiltration) prior to application to chromatography media.

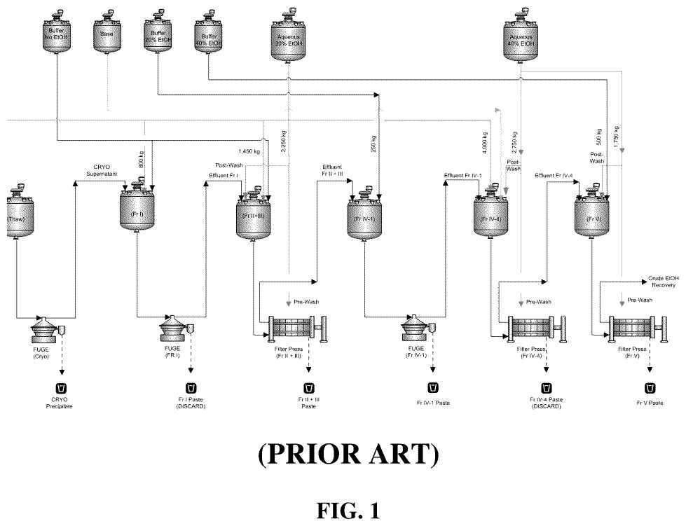

It should be appreciated that prior art methods for industrial scale protein isolation from blood products such as serum, plasma, and cryo-poor plasma (i.e., Cohn or Kistler and Nitschmann processes) have used ethanol to isolate proteins by precipitation. A typical process flow for the Cohn ethanol fractionation process for isolation of proteins from plasma is shown in FIG. 1. As shown, in the Cohn ethanol fractionation process ethanol is added to the supernatant generated from plasma by cryoprecipitation (i.e., cryo-poor plasma) to generate Fraction I. The supernatant from Fraction I is subsequently treated with additional ethanol to produce Fraction II/III. Supernatants from this and subsequent fractions are treated with more ethanol to generate Fraction IV-1, Fraction IV-4, and Fraction V. Due to heat generated on mixing of ethanol with water the addition of ethanol needs to be gradual and (for reasons detailed below) requires stringent, sub-zero Celsius, temperature control. Accordingly, each ethanol addition step represents a significant process bottleneck. As a result, ethanol precipitation methods performed at an industrial scale typically require 5 to 7 days to generate a useable IgG preparation from plasma, require the use of complex temperature-controlled facilities, and have high power requirements for refrigeration. In addition, ethanol-containing waste products can require specialized disposal, additional energy, and costs.

As noted above, since dilution of ethanol into an aqueous protein solution generates considerable heat it is necessary to apply temperature control in the form of refrigeration throughout ethanol precipitation methods for isolating proteins from plasma in order to avoid protein denaturation. Because the Cohn process operates at near cryogenic temperatures, energy directed to cooling and the associated costs make a major contribution to the environmental impact and cost of producing the protein product (e.g., IgG). The cooling-energy cost can be grouped into process related (prechilling of ethanol to −20° C. plus chilling of buffers) and facility related (e.g., need for expansive cold rooms). It should also be appreciated that the problem of removing excess heat becomes considerably more complex as the process is scaled up. In addition, ethanol is generally recovered from waste streams of the Cohn process in an attempt to reduce environmental impact and further impacting overhead costs and energy consumption.

As such, prior art industrial scale processes for protein isolation from such blood-based materials are very energy intensive. For ethanol recovery by distillation, the distillate is cooled by the column overhead condenser to approximately ambient temperature and is reconstituted with ambient temperature virgin ethanol. However, to reuse the ethanol in the process it must be cooled to −20° C. The refrigerant energy consumption required to chill the ethanol from ambient to −20° C. is almost 1 million BTU per 10,000 L of plasma. Energy requirements (as estimated in an engineering study performed by Hughes Engineering Consulting) are provided in Table 1.

TABLE 1

| Cohn | Per 10,000 L | Per 5 × 10⁶ L | Per 10⁷ L | Per 1.5 × 10⁷ | Per 2 × 10⁷ |
|---|---|---|---|---|---|
| (ethanol) | plasma (mm | plasma (mm | plasma (mm | L plasma | L plasma |
| Process | BTU) | BTU) | BTU) | (mm BTU) | (mm BTU) |
| Reboiler | 40.1 | 20046.0 | 40092.0 | 60138.0 | 80148.0 |
| energy | | | | | |
| Condenser | 39.0 | 1965.0 | 39300 | 5985.0 | 78600.0 |
| energy | | | | | |
| Ethanol/Buffer | 54.7 | 27333.0 | 54666.0 | 81999.0 | 109332.0 |
| chilling | | | | | |
| Cold Room | 25.8 | 12923.3 | 25846.7 | 38770.0 | 51693.3 |
| chilling | | | | | |
| Total energy | 159.6 | 79952.3 | 159904.7 | 239857.0 | 319809.3 |
| use | | | | | |

In addition to adding to energy requirements, the addition of ethanol raises the issue of removal of this organic solvent from the protein-containing solutions so produced. While a certain amount of ethanol can be recovered from these solutions by processes such as diafiltration and/or distillation of the solution passing through the diafilter, the formation of alcohol/water azeotropes makes it inevitable that large amounts of ethanol-containing waste is generated. As such, prior art at-scale processes based on ethanol precipitation generate a significant amount of organic waste.

On the other hand, salts or organic polymers utilized in the methods described above can be easily recovered following simple evaporation of water from the waste solutions so produced. It should be appreciated that the dissolution of salts or hydrophilic polymers as described above does not generate heat, and as such these processes can be performed at ambient temperature using conventional equipment, without the need for refrigeration of the manufacturing rooms.

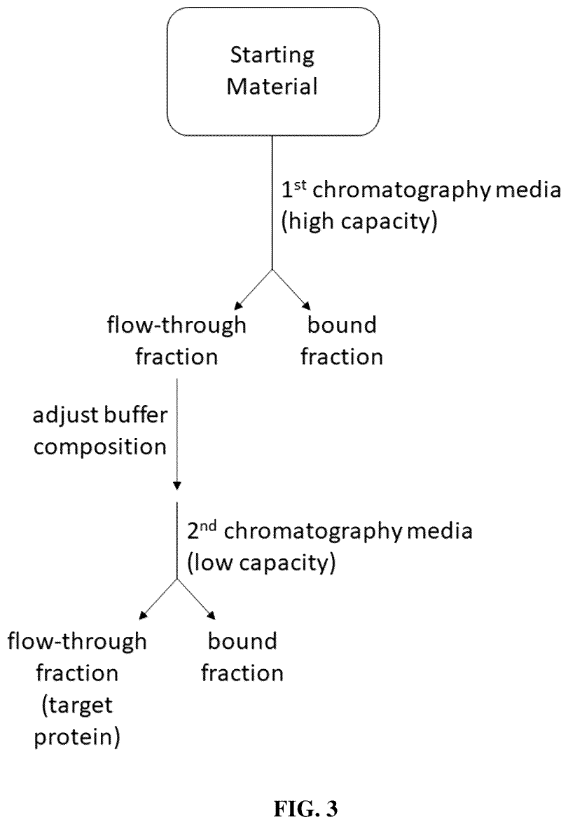

Methods of the inventive concept avoid many of these issues. An example of a method of the inventive concept is shown schematically in FIG. 3. As shown, following one or more precipitation steps applied to a blood produce (such as serum, plasma, cryo-poor plasma, cryo-poor plasma into which the cryoprecipitate has been redissolved, etc.) and utilizing the addition of an organic salt (such as a citrate or acetate salt), a solution containing the protein of interest and one or more contaminants is first applied to a high capacity chromatography media, where the protein of interest is maintained in the flow-through fraction. This flow-through fraction is then applied to a low-capacity chromatography media, which can utilize a different separation mode than the high-capacity chromatography media. For example, the high capacity and low-capacity chromatography media can be ion exchange media with opposite charges (e.g., cation exchange vs anion exchange). Buffer conditions (e.g., ionic strength, pH, temperature, etc.) can be adjusted prior to this application. Buffer conditions can be adjusted by any suitable means, for example addition or removal of one or more salt(s), addition of acid or base, etc. Suitable media include, but are not limited to, ion exchange media (e.g., DEAE, Q. S, or CM media), hydrophobic interaction media (e.g., propyl, butyl, or phenyl media), affinity media, or mixed-mode media.

As noted above, the functionality of the chromatography media is selected to interact with both the protein of interest and one or more contaminants, however the capacity of the media is selected to match or slightly exceed (e.g., by 1%, 5%, 10%, 25%, 50%, 100%, 150%, or 200%) the capacity at which breakthrough of contaminant occurs. The protein of interest is subsequently collected at high yield and purity (as described above) in the flow-through fraction. For many proteins, such as IgG, these steps can be performed at ambient temperature and within a relatively short period of time (less than 30 minutes, less than 1 hour, less than 2 hours, less than 3 hours, or less than 4 hours per step).

It should also be appreciated that retention of the protein of interest in flow-through fractions generated by chromatography steps can significantly reduce the amount of chromatography media required (relative to prior art methods involving binding and elution of the target protein from chromatography media. In methods of the inventive concept a first chromatography media can be selected to bind a first contaminant or set of contaminants. If previous processing steps, such as selective precipitation, have been applied a large portion of contaminating proteins and other compounds will have been removed prior to such an initial chromatography step. As such, the volume of such a first chromatography media can be scaled to bind a portion of the already reduced contamination. In addition, as noted above media utilized in a second chromatography step is minimized to reduce or eliminate loss of the target protein. Accordingly, industrial scale methods of the inventive concept can utilize relatively small amounts of chromatography media, reducing the footprint of the facility used, permitting use of a wider range of chromatography media (which may not require selection on the basis of mechanical properties), and reducing overall processing time.

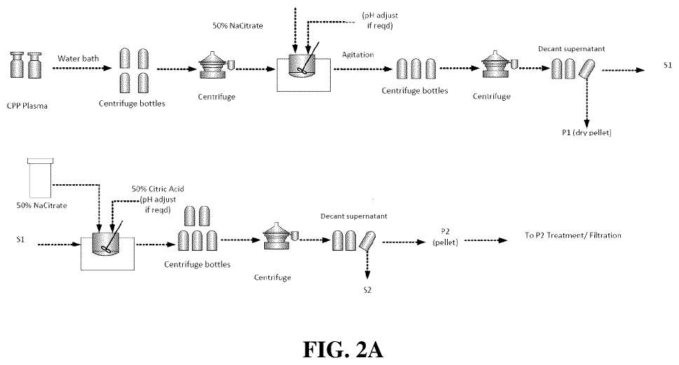

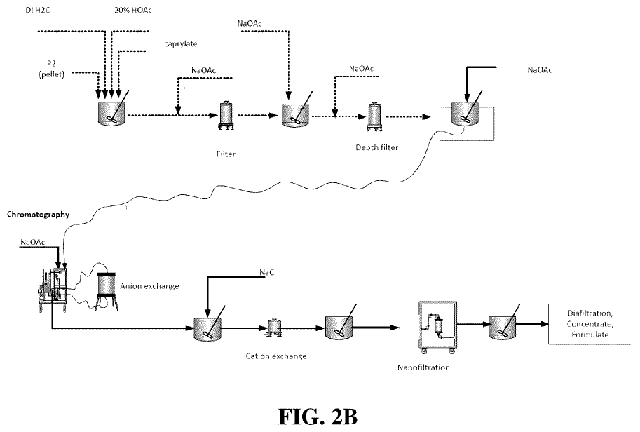

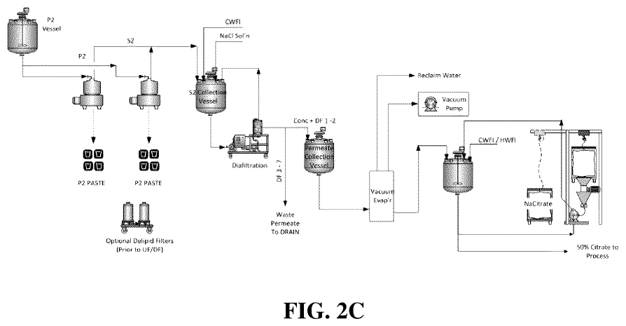

A typical process flow for at scale processing for isolation of IgG from frozen plasma using a method of the inventive process is shown in FIGs. B1 and B2. As shown in FIG. 2A, frozen plasma (e.g., frozen plasma units obtained from a plasma collection center) is thawed, transferred to containers suitable for centrifugation, and centrifuged to remove the cryoprecipitate. Alternatively, a flow through centrifuge can be used to separate the cryoprecipitate from the liquid cryo-poor plasma (CPP). A concentrated citrate salt solution is then added to the CPP while mixing to generate a suspension containing the first precipitate (P1) and the first supernatant (S1). This suspension is transferred to centrifuge-compatible containers and centrifuged to separate P1 from S1. Alternatively, a flow-through centrifuge or filter press can be used. For IgG isolation, S1 is further processed. The P1 portion can be discarded or, preferentially, further processed to isolate other commercially valuable proteins. As shown in FIG. 2A, S1 is further treated by the addition of a concentrated citrate salt stock solution while mixing to generate a second suspension containing a second precipitate (P2) and a second supernatant (S2). This second suspension is transferred to centrifuge-compatible containers and centrifuged to separate P2 (which contains IgG) from S2. Alternatively, a flow-through centrifuge or filter press can be used. S2 can be discarded or, preferentially, further processed to recover the citrate salt for re-use (as described below) and/or further processed to recover additional commercially valuable proteins.

Further processing of P2 to isolate IgG in an at-scale process is shown in FIG. 2B. As shown, P2 is resuspended and dissolved in water and transferred into an acetate buffer (which can have a slightly acidic pH of about 5 to 6.9, preferably about 5.7). Caprylate is then added for virus inactivation and undissolved material removed using a filter. Solid materials remaining after or generated by caprylate treatment are removed using a depth filter, and the resulting solution subjected to anion exchange chromatography. Additional acetate buffer can be added to adjust protein concentration. If necessary, multiple anion exchange media devices (such as chromatography columns) can be used in parallel and the flow through fractions pooled and mixed prior to application to cation exchange media. In methods of the inventive concept the capacity of this cation exchange media is low, and while conditions are selected such that IgG can bind to the cation exchange media a high percentage (e.g. 70%, 80%, 90%, or more) of the IgG content of the starting plasma material is recovered in the flow through from the cation exchange media. If necessary, more than one cation exchange media device (such as a chromatography column) can be used in parallel and the flow through fractions collected and mixed prior to nanofiltration to remove viral particles. If necessary, more than one nanofiltration device can be used and the permeates collected and pooled before buffer exchange (e.g., by diafiltration) and concentration to the desired value for pharmaceutical use (e.g., from 1% to 20% by weight).

It should be appreciated that such a process flow provides a linear path for isolation of IgG from plasma, and that process bottlenecks can be readily addressed through the use of appropriate hardware (e.g., flow through centrifuges) and appropriate sizing (or, if necessary, duplication) of chromatography appliances. Chromatography process steps can be carried out at ambient temperatures, and the use of flow through fractions in chromatography steps avoids the need to halt production to provide for washing and elution of the chromatography media (as in prior art chromatography-based methods).

It should be appreciated that such an approach can greatly simplify processing of protein-containing solutions at large volumes (e.g., greater than 2 L, 10 L, 50 L, 100 L, 200 L, 250 L, 1,000 L, 2,500 L, 5,000 L, 8,000 L, 10,000 L, 20,000 L, or more), as collection of the protein of interest does not require a separate set of elution steps and the process utilizes a minimal amount of chromatography media. Within the context of this application industrial scale refers to processing of from about 200 L (e.g., for specialty antisera) to 20,000 L (e.g., for nonspecific immunoglobulins) of a protein-containing solution (e.g., serum, plasma, cryo-poor plasma, cryo-poor plasma in which cryoprecipitate has been re-dissolved, etc.).

As noted above, preferred embodiments of the inventive concept can utilize two different chromatography media. One of these is selected to not interact with the protein of interest, and to bind a portion of the contaminating species present. The second chromatography media differs from the first chromatography media, and is selected and used under conditions in which it can bind with the protein of interest. Inventors have surprisingly found that such second chromatography media can be employed in negative selection mode (i.e., with the protein of interest being recovered in the flow-through from the media) to provide simple, efficient, and highly scalable methods.

In methods of the inventive concept the capacity of the second chromatography media (which is a function of both the chemistry of the chromatography media, the chromatography buffer, and amount used) is selected on the basis of its ability to bind contaminants remaining in the flow-through obtained from the first chromatography media. This capacity can, for example, be determined using break-through studies in which contaminants are applied to the second chromatography media until they appear in the flow-through (under defined conditions of buffer composition and flow rate). An amount of the second chromatography media sufficient to bind contaminant content at the desired scale can then be utilized in a combined process using both the first and second chromatography media. In some embodiments a small excess of capacity in this second chromatography media (e.g., 10%, 20%, 30%, 40%, 50%, 60%, 70%, 80%, 90%, or 100% excess) can be used.

Notably, the capacity of the second chromatography media is significantly less than that used in a conventional isolation method in which the protein of interest is bound and subsequently eluted (e.g., less than 10%, 5%, 2.5%, 1%, 0.5%, 0.25%, 0.1%). In some embodiments this permits use of functionalized (e.g., charged) filters having appropriate pendant groups in place of traditional bead or particle-based chromatographic media. This advantageously permits combining particle removal and chromatography steps.

Inventors have found that, despite having the ability to bind with the target protein, methods of the inventive concept typically recover the protein of interest at high yield (in excess of 60%, 70%, 75%, 80%, 85%, 90%, or 95%) in the flow-through from the second chromatography media. Without wishing to be bound by theory, the Inventors believe that utilization of a second chromatography step that utilizes a judiciously minimized amount of media capacity results in displacement of the protein of interest from the media by contaminants present in the first flow-through. This removes the need for an elution step, and results in increased yield. In addition, collection of the protein of interest in successive flow-through fractions from successive chromatography steps greatly facilitates and simplifies the isolation process and transition from bench scale (up to about 2 L of starting material) to industrial scale (200 L to 20,000 L or more of starting material).

One should appreciate that the disclosed techniques provide many advantageous technical effects including rapid provision of proteins from complex solutions at high purity and high yield at process scale. An exemplary time course for isolation of IgG from frozen plasma that has been thawed to generate cryoprecipitate is shown in Table 2.

TABLE 2

| I. Step | Time Elapsed (min) |
|---|---|
| Cryoprecipitate removal (centrifugation) | 40 |
| First addition of citrate salt | 60 |
| Separation of first supernatant and first precipitate (centrifugation) | 60 |
| Second addition of citrate salt | 120 |
| Separation of second supernatant and second precipitate (centrifugation) | 60 |
| Removal of water from second precipitate | 60 |
| pH and caprylate treatment of resuspended second precipitate | 45 |
| Filtration of treated resuspended second precipitate | 45 |
| Dilution and conductivity adjustment | 10 |
| Filtration to remove particulates | 90 |
| Anion exchange | 280 |
| Cation exchange | 130 |
| Nanofiltration | 290 |
| Concentration for formulation (low pressure* diafiltration) | 201 |
| Total | 1491 minutes |
| | (24.85 hours) |

As shown, isolation from a crude starting material to sterile, concentrated (10-20 mg/mL or higher) IgG at high purity can be performed in about 24 to 48 hours. Process times of 36 hours, 48 hours, and 72 hours are contemplated. For example, process model studies indicate that IgG isolation from 5,000 L of frozen plasma can be completed in less than 48 hours, particularly when precipitating agents are added as dry or crystalline solids. Inventors have found that addition of the precipitating agent as a dry or crystalline solid instead of a 50% stock solution can reduce the processing time for isolation of IgG from the crude starting material to less than 24 hours. This is in contrast to the 5 to 7 days typical for the Cohn ethanol precipitation process. Such short processing times permit a more rapid turnaround in processing large amounts of starting material. In addition, it should be appreciated that such short process times when coupled with higher yields that are realized by methods of the inventive concept (typically 70%, 80%, 90%, 95%, or more) relative to Cohn ethanol fractionation (typically 40% to 50%) provide for a 2-fold, 3-fold, 4-fold, 5-fold or more increase in productivity and thereby operating-cost savings, using methods of the inventive concept

The Inventors' process can produce a more native (i.e., less denatured) protein than prior art methods, since the protein is not exposed to an organic solvent, is not bound and subsequently eluted from chromatography media using harsh conditions, and is not processed for an extended period of time. This advantageously both enhances yield and reduces the chance of denaturation, while also simplifying the isolation process and greatly reducing processing time. As such it is distinct and different from (and significantly more cost effective than) current protein isolation processes that involve binding to and elution from chromatographic media, with improved protein stability, increased in vivo half-life, more rapid infusion rates, improved patient tolerance, and reduced immunogenicity compared to conventionally produced protein therapeutics.

First and second chromatography media can be selected to have complementary binding characteristics. For example, the first chromatography media and the second chromatography media can be ion exchange media that have opposing charges under the separation conditions used (e.g., anion exchange followed by cation exchange, cation exchange followed by anion exchange). Although examples provided below cited the use of ion exchange media, Inventors contemplate that any chromatographic media having complementary binding characteristics can be paired in methods of the inventive concept. Suitable chromatography media include, but are not limited to, ion exchange media (e.g., DEAE, Q, CM, and/or S chromatography media), hydrophobic interaction chromatography media, affinity chromatography media, and mixed-mode chromatography media. For example, in some embodiments first and second chromatography media can be ion exchange media with opposing charges under separation conditions. Alternatively, in some embodiments ion exchange chromatography media or mixed-mode chromatography media can be paired with hydrophobic interaction chromatography media (with appropriate adjustments to ionic strength hydrophobicity??? between chromatographic steps). In still other embodiments, an affinity chromatography media can be used as a first chromatographic media and ion exchange, hydrophobic interaction, or mixed-mode chromatographic media as the second chromatography media.

Preferred embodiments of the inventive concept can utilize both anion and cation exchange chromatography (which are relatively inexpensive and available in a wide variety of formats), where buffer conditions and column binding capacity are selected or optimized to provide the target protein (e.g., IgG) in the flow-through fraction of each chromatographic step. Towards this end an initial ion exchange step (e.g., anion exchange, cation exchange) can be performed using a high-capacity ion exchange media that does not appreciably bind the protein of interest.

For example, in the isolation of IgG a large/high-capacity anion exchange step can be performed, providing a flow-through fraction containing IgG (along with some contaminants) and retaining a bound fraction that includes a portion of the contaminating proteins present in the starting material. This flow-through fraction is then applied (in some embodiments following an adjustment of salt content and/or pH to adjust ionic strength/conductivity and or pH) to a small or low-capacity cation exchange media.

The size of this small or low capacity cation exchange media is selected so that it is near or slightly (e.g., 1%, 2%, 5%, 10%, 20%, 30%, 40%, 50%) greater than the amount or capacity for breakthrough of a contaminating protein found in the flow-through of the anion exchange media (taking buffer conditions into account). Without wishing to be bound by theory, the Inventors believe that this permits contaminating proteins to displace IgG that may bind to the cation exchange media. Careful selection of the amount/capacity of the cation exchange media and/or buffer conditions provides efficient removal of contaminating protein while also providing high yields of IgG.

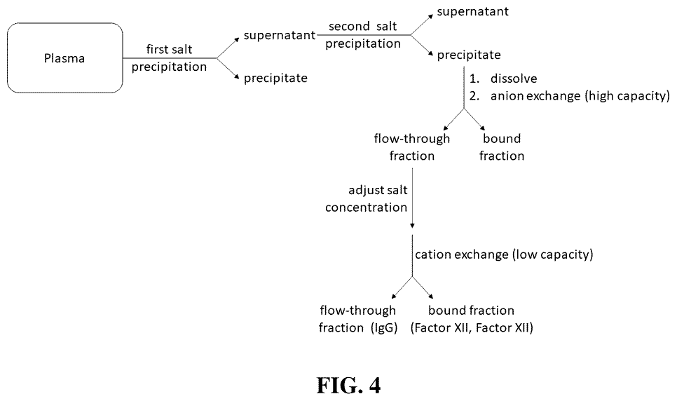

An example of a process of the inventive concept is shown in FIG. 4. It should be appreciated that in this context a starting material can be serum, plasma, cryo-poor plasma, cryo-poor plasma into which the cryoprecipitate has been re-dissolved, or a fraction (e.g., a supernatant or a dissolved precipitate) resulting from a precipitation step applied to such materials. Although blood proteins are of interest, a suitable starting material can be any solution containing a protein of interest (e.g., cell cultures, supernatants or lysates of cells from mammalian, bacterial, fungal, insect, or plant-based tissues or tissue cultures, solvated inclusion bodies, animal egg contents, milk, urine, or other body fluids, etc.). As shown, a starting material is applied to a high capacity first chromatography media selected to bind a portion of the contaminants and to not bind the protein of interest. The flow-through of this first chromatography step is directed (in some instances, after an adjustment in buffer composition and/or pH) to a second, different chromatographic media that is provided at low capacity. This second chromatography media can potentially bind the protein of interest as well as contaminants remaining in the first flow-through, but the capacity of the media is selected such that available binding sites are occupied by contaminants. Under these conditions the target protein is recovered in the flow-through from the second chromatography step at high yield (e.g., with less than 30%, 25%, 20%, 15%, 10%, 5%, 2.5%, or 1% of target protein present in the first flow-through lost).

Methods of the inventive concept are particularly suitable for isolation of therapeutic proteins, for example from blood and blood products. Examples of blood products include serum, plasma, cryoprecipitate, cryo-poor plasma, and cryo-poor plasma into which cryoprecipitate has been re-dissolved. Similarly, methods of the inventive concept are suitable for isolation of therapeutic proteins from products of conventional processes for protein isolation from blood products, including precipitation (e.g., with an organic solvent, an inorganic salt, an organic acid salt, and/or a hydrophilic polymer), chromatography, ultrafiltration, and/or diafiltration.

Methods of the inventive concept can also be applied to non-blood sources of therapeutic proteins. These include cell cultures, supernatants or lysates of cells from mammalian, bacterial, fungal, insect, or plant-based tissues or tissue cultures, solvated inclusion bodies, animal egg contents, milk, urine, or other body fluids, etc. products of cell-free protein synthesis, and products of protein conjugation processes.

Therapeutic proteins towards which methods of the inventive concept can be applied include, but are not limited to, albumin, alpha-one antitrypsin, immunoglobulins (e.g., IgG, IgM, IgA, IgY), clotting factors (e.g., Factor VIII, von Willebrand Factor), and host cell proteins (HSP).

Inventors have found methods of the inventive concept are particularly useful in the isolation of IgG from blood plasma, although application to other solutions containing IgG (e.g.. cell culture media, cell lysates, other body fluids, etc.) is contemplated. Within the context of this application plasma is considered to include freshly collected plasma, refrigerated plasma, frozen plasma, cryo-poor plasma, and cryo-poor plasma into which the cryoprecipitate has been re-dissolved. Such plasma can, for example, be obtained as pooled material from commercial collection centers.

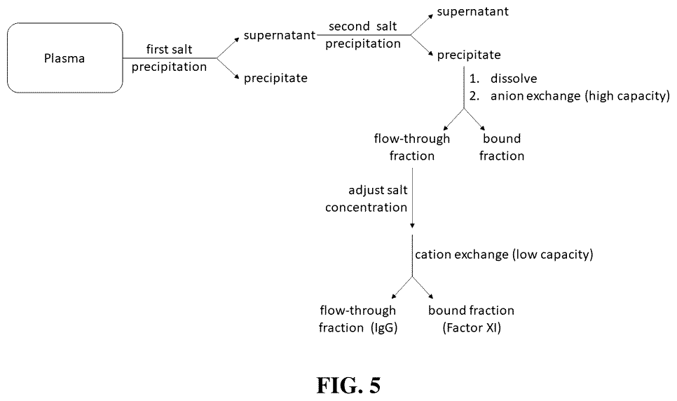

An example of a method of the inventive concept for isolation of IgG (which can be applied to a process flow as shown in FIGs. B1 and B2) from plasma is shown schematically in FIG. 5. The Inventors used two salt precipitation steps (i.e., about 11% in the first salt precipitation and about 26% in the second salt precipitation) to generate a protein solution from cryo-poor plasma (CPP). This protein solution served as starting material for the subsequent chromatography step and contained maximum IgG yield and minimized concentrations of unwanted proteins. As shown, the first precipitation step produces an IgG-rich supernatant, and the second precipitation step produces an IgG-rich precipitate or paste. This IgG-rich precipitate is dissolved (e.g., in water) prior to ion exchange steps. As noted below, in preferred embodiments a buffer exchange step (e.g., dialysis, diafiltration, ultrafiltration followed by dilution, size exclusion chromatography, etc.) is not performed prior to ion exchange steps.

It should be appreciated that citrate salts and similar precipitating agents can be recovered from intermediate products of methods of the inventive concept (such as the second supernatant) using relatively simple methods. For example, citrate can be recovered by diafiltration and readily concentrated by low energy methods such as evaporation. Such methods can recover up to 90% of organic acid salts (such as citrate salts) utilized in such methods. Such recovered organic acid salt can be supplemented with a small amount of new material and recycled into the process, thereby reducing both material costs and ecological impact. A typical process for recovering citrate salts from an IgG isolation method is shown in FIG. 2C. As shown, supernatant collected from the second precipitation step (S2) as well as liquid removed from the precipitate (P2) from the second precipitation step can be collected and subjected to diafiltration. The permeate (containing the dissolved organic salt) is transferred to a vacuum evaporator to remove water (which can be reclaimed) and generate a concentrated solution of the salt. This can be supplemented with a small amount of organic acid salt to generate a 50% stock solution that can be re-introduced into precipitation steps of the process. It should be appreciated that these steps are relatively low energy steps that do not require the application of heat. This is in contrast to the processes required to recover ethanol from intermediate products of ethanol precipitation methods such as the Cohn process.

As shown in FIG. 5, using IgG as an example, the isolation process of the target protein in the inventive concept provides a first ion exchange step that can be performed using an anion exchange media (e.g., DEAE or Q chromatography media). In some embodiments a strong anion exchange media can be used that maintains a positive charge over a wide range of pH conditions (e.g., pH 1 to 14, pH 2 to 13, pH 3 to 12, pH 4 to 11). Chromatography media (e.g., anion exchange media as cited in this example) can be provided in any suitable form and/or on any suitable support (e.g., agarose, cross linked agarose, cellulose, polyacrylamide, polystyrene, glass, or combinations thereof) and in any suitable configuration (e.g., porous beads, non-porous beads, fibers, wools, filters, etc.).

As shown in FIG. 5, in an IgG process of the inventive concept IgG is recovered in the flow-through (i.e., unbound) fraction from the anion exchange media. This first flow-through fraction is subsequently applied to a small or low capacity cation exchange media (e.g., media that includes carboxylate or sulfonate groups) in which size/capacity has been selected such that contaminants (e.g., Factor XI, activated Factor XI, Factor XII, activated Factor XII) are retained while IgG (which typically also binds to cation exchange media) passes through in the flow-through fraction (i.e., the second flow-through fraction). Typically, the capacity or size of the cation exchange media is selected to be at or slightly exceeding (e.g., by 1%, 2%, 5%, 10%, 15%, 20%, 25%, 30%, 50%, 75%, or 100%) its capacity for contaminants present in the first flow-through fraction. Without wishing to be bound by theory, Inventors believe that when a cation exchange media is so selected bound protein (e.g., IgG) is displaced by the contaminating protein and released into the second flow-through fraction, thereby increasing yield. In preferred embodiments loss of IgG in the cation exchange step is less than about 30%, 25%, 15%, 10%, 7.5%, 5%, 4%, 3%, 2%, or 1% of IgG content of the starting material.

Such second chromatography media (e.g., a cation exchange media in this example) can be provided on any suitable support (e.g., agarose, cross linked agarose, cellulose, polyacrylamide, polystyrene, glass, or combinations thereof) and in any suitable configuration (e.g., porous beads, non-porous beads, fibers, wools, filters, etc.). Inventors have found that the capacity of the cation exchange media utilized can be quite small, and in preferred embodiments can be provided by a filter with pendant cation exchange groups. This advantageously combines purification and clarification steps.

In some embodiments of the inventive concept the buffer composition and/or pH of the first flow-through fraction can be modified prior to application to the second chromatography media (e.g., the cation exchange media of this example) in order to optimize capacity and selectivity for contaminants. For example, buffer conditions can be selected or adjusted such that contaminants have a higher affinity for the second chromatography media than that of the target protein. In preferred embodiments of protein (IgG) isolation methods of the inventive concept salts can be added to increase the ionic strength or conductivity of the first flow-through fraction to fall within a desired range. Alternatively, the first flow-through can be diluted or subjected to buffer exchange (e.g., dialysis, diafiltration, etc.) prior to application to the second chromatography media.

In some embodiments the second flow-through fraction can be subjected to additional processing steps. Such additional processing steps can include nanofiltration for virus removal, such as filtration using a 0.02 μm pore membrane. Inventors have found that this effectively retains any remaining virus particles while minimizing yield losses.

In some embodiments the purified protein solution can be prepared for use by concentration and diafiltration in order to provide a drug product having a useful concentration in a pharmacologically compatible buffer that provides stability. For IgG, such a step can provide an IgG concentration of about 5% IgG (w/v) in a suitable formulation buffer (e.g., 0.2M glycine pH 4.2 to pH 6.5) with minimal losses. Concentration can be increased or otherwise adjusted using known methods.

Typical IgG yield results for an IgG isolation process of the inventive concept are shown in

Table 3.

TABLE 3
Total yield based on A₂₈₀ absorbance/initial nephelometry values
corrected for sample removal. Extinction Coefficient for IgG: 1.3

| | | | | | Total IgG | IgG | Total IgG | IgG |
|---|---|---|---|---|---|---|---|---|
| | Concentration | Volume | | Total | [A280] | [Neph] | [Neph] | Yield |
| Sample | (A280/mL) | (mL) | | (A280) | (mg) | (mg/mL) | (mg) | (%) |
| Cryo-poor | 44.41 | 2000 | | 88820 | N/A | 8.25 | 16500 | 100.0 |
| Plasma | | | | | | | | |
| First | 35.62 | 2490 | | 88693.8 | | 6.34 | 15786.6 | 95.7 |
| Supernatant | | | | | | | | |
| First | N/A | 22.9 | g | N/A | | N/A | | |
| Precipitate | | | | | | | | |
| Second | 14.07 | 3850 | | 54169.5 | | | | |
| Supernatant | | | | | | | | |
| Second | N/A | 187 | g | N/A | | | | |
| Precipitate | | | | | | | | |
| Dissolved | 14.73 | 2000 | | 29460 | N/A | 7.33 | 14660 | 88.8 |
| Second | | | | | | | | |
| Precipitate | | | | | | | | |
| Post Depth | 10.47 | 2570 | | 26907.9 | N/A | 5.86 | 15060.2 | 91.3 |
| Filter | | | | | | | | |
| Post 0.2 μm | 6.37 | 4284 | | 27289.08 | N/A | 3.49 | 14951.16 | 90.6 |
| Filter | | | | | | | | |
| Anion | 2.63 | 5680 | | 14938.4 | N/A | 2.3 | 13064 | 79.2 |
| Exchange | | | | | | | | |
| Flow- | | | | | | | | |
| Through | | | | | | | | |
| Cation | 2.52 | 5930 | | 14943.6 | 11495 | 2.13 | 12630.9 | 76.6 |
| Exchange | | | | | | | | |
| Flow- | | | | | | | | |
| Through | | | | | | | | |
| Post Nano- | 2.24 | 6107 | | 13679.68 | N/A | N/A | | |
| Filtration | | | | | | | | |
| Final Product | 67.11 | 248 | | 16643.28 | 12802 | N/A | N/A | 77.6 |

| | |
|---|---|
| Total Additive Loss from Sample Removal | 0.96 |

Test results obtained from the material shown in Table 3 (i.e., 44.94 mg/mL IgG) are shown in Table 4.

| Test | Result | | Pass/Fail | EP Standard | |
|---|---|---|---|---|---|
| PKA | 1.48 | IU/mL | Pass | <35 | IU/mL |

| | | | | |
|---|---|---|---|---|
| IgA | 1.57 | μg/mL | N/A | “Not more than is stated on the product |
| | | | | label” |

| IgM | | Not Detectable | | N/A | | No EP standard |
|---|---|---|---|---|---|---|
| IgG subclass | | IgG 1 | IgG 2 | IgG 3 | IgG 4 | Product should be |
| | Starting Material | 62 | 28 | 7 | 4 | representative of |
| | Finished Product | 62 | 30 | 7 | 1 | starting material |

| Fc function | 119% | | Pass | >60% | |
|---|---|---|---|---|---|
| ACA | 0.83 | CH50U/mg | Pass | ≤1.5 | CH50U/mg |
| NaPTT | 219.5 | sec | Pass | >200 | sec |

| | | | | |
|---|---|---|---|---|
| FXIa (chromogenic) | <0.04 | mU/mL | Pass | No EP standard |
| FXIa (eCAT/TGA) | 0.79 | mU/mL | Pass | No EP standard |
| | | | | (<1 mU/mL historically |
| | | | | in prior art) |

In addition to remarkably low levels of Factor XI contamination, the Inventors have found that methods of the inventive concept provide surprisingly low levels of IgA contamination.

Methods of the inventive concept applied to IgG were found to provide robust and commercially scalable processes that consistently produce about a 72-78% or higher yield of IgG from starting plasma in as little as about 24, 36, or 48 hours. The resulting product is >99% pure IgG product with 100% functionality.

As noted above, methods of the inventive concept provide considerable energy savings over prior art ethanol precipitation-based processes such as Cohn precipitation. Energy cost estimates for Cohn ethanol precipitation for various volumes of plasma are provided in Table 5. Estimated energy required to process the same volumes of plasma using a method of the inventive concept is provided in Table 6.

TABLE 5

| Prior Art Cohn | Per 10,000L | Per 5 × 10⁶ L | Per 10⁷ L | Per 1.5 × 10⁷ | Per 2 × 10⁷ |
|---|---|---|---|---|---|
| (ethanol) | plasma (mm | plasma (mm | plasma (mm | L plasma | L plasma |
| Process | BTU) | BTU) | BTU) | (mm BTU) | (mm BTU) |
| Reboiler | 40.1 | 20046.0 | 40092.0 | 60138.0 | 80148.0 |
| energy | | | | | |
| Condenser | 39.0 | 1965.0 | 39300 | 5985.0 | 78600.0 |
| energy | | | | | |
| Ethanol/Buffer | 54.7 | 27333.0 | 54666.0 | 81999.0 | 109332.0 |
| chilling | | | | | |
| Cold Room | 25.8 | 12923.3 | 25846.7 | 38770.0 | 51693.3 |
| chilling | | | | | |
| Total energy | 159.6 | 79952.3 | 159904.7 | 239857.0 | 319809.3 |
| use | | | | | |

TABLE 6

| Citrate salt | Per 10,000 L | Per 5 × 10⁶ L | Per 10⁷ L | Per 1.5 × 10⁷ | Per 2 × 10⁷ |
|---|---|---|---|---|---|
| precipitation | plasma (mm | plasma (mm | plasma (mm | L plasma | L plasma |
| Process | BTU) | BTU) | BTU) | (mm BTU) | (mm BTU) |
| Heat energy | 18.6 | 1311.2 | 2622.5 | 3933.7 | 5244.9 |
| (citrate | | | | | |
| reclamation) | | | | | |
| Chilling/cooling | 0.0 | 0.0 | 0.0 | 0.0 | 0.0 |
| energy | | | | | |
| Total energy | 18.6 | 1311.2 | 2622.5 | 3933.7 | 5244.9 |
| use | | | | | |

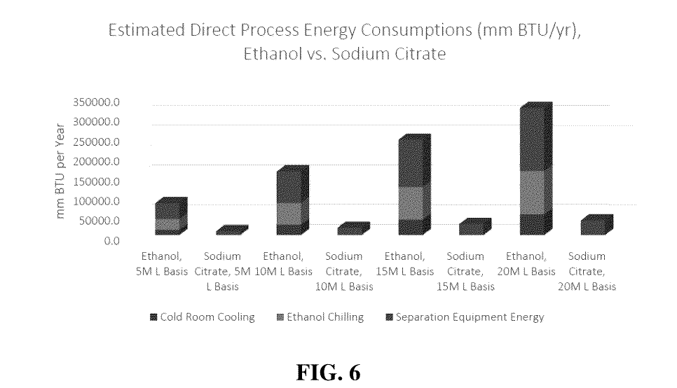

As shown, estimated energy costs for a method of the inventive concept represent an approximately 89% reduction in energy consumption relative to the prior art Cohn fractionation process at the same scale. These differences are shown in terms of estimated annual energy consumption in FIG. 6. This reduced energy consumption not only reduced costs for production, but also reduces the carbon footprint of the process. It should be appreciated that improved efficiency in the recovery of IgG using methods of the inventive concept (i.e., about 70% to 90% or more) provide still greater energy savings and carbon footprint reduction on a per gram of IgG produced basis relative to prior art ethanol precipitation methods (typically 40% to 50%).

It should be appreciated that additional proteins can be recovered from various intermediate product streams produced by methods of the inventive concept, for example the first precipitate (P1), the second supernatant (S2), the bound fraction from the first chromatography step, and/or the bound fraction from the second chromatography step. Such intermediate products can be treated by any suitable method (e.g., additional precipitation steps, affinity chromatography, size exclusion chromatography, hydrophobic interaction chromatography, ion exchange chromatography, and/or mixed mode chromatography) to facilitate isolation of additional non-IgG proteins from the starting material.

The IgG isolation methods described above can utilize two citrate precipitation steps. The first of these produced a first supernatant (S1) containing all or most of the IgG and albumin present in a starting plasma raw material. Additional citrate is added to this first supernatant to produce a second supernatant (S2) and a second precipitate (P2). Essentially all of the IgG is found in P2, whereas essentially all of the albumin remains in S2. Inventors have found that S2 can be further processed to provide albumin at high yield (e.g., 70% to 90% or higher) and high purity (e.g., 90% to 98% or higher) using chromatography steps in which the albumin is retained in the flow-through fraction. As in isolation of IgG, this advantageously reduces processing time and the amount of chromatography media provided. In addition, such processing of the S2 supernatant can provided for an integrated method for isolation of both IgG and albumin from frozen plasma at high purity (e.g., greater than 90%, 95%, 98%, 99% or higher) and yield (e.g., greater than 70%, 80%, 85%, 90%, 95% 98% 99%, or higher) for both target proteins. In some embodiments albumin so produced shows no other proteins detectable by SDS-PAGE analysis.

In some embodiments of the inventive concept, methods for isolating albumin from the second supernatant of an IgG isolation method as described above (i.e., S2) can include additional of a precipitating agent to the second supernatant. Such a precipitating agent can be a salt (such as a sulfate, phosphate, or chloride), a salt of an organic acid (such as a citrate or acetate salt), an organic acid or a salt thereof (such as C4 to C10 carboxylic acid), a hydrophilic polymer (such as dextran, polyethylene glycol, etc.), and/or an organic solvent (e.g., ethanol, acetone, etc.). Such a precipitating agent can be added in an amount effective to cause precipitation of albumin from the S2 fraction (e.g., from 1% w/v to 50% w/v). Alternatively, such a precipitating agent can be added in an amount effective to cause precipitation of contaminants (e.g., undesired proteins) from S2 while retaining albumin in solution (e.g., from 1% w/v to 50 w/v). Such effective amounts can be readily determined by addition of the precipitating agent, allowing a third precipitate (i.e., P3) and a third supernatant (i.e., S3), and characterizing the contents of S3 and P3 by any suitable means (e.g., SDS-PAGE, capillary electrophoresis, immunoassay, mass spectrometry, etc.). Such a precipitation reaction can be carried out any suitable temperature. Preferably such a precipitation can be carried out at ambient temperature or at an elevated temperature (e.g., 30° ° C., 35° C., 40° C., 45° C., 50° ° C., 55° ° C., 60° ° C., 65° C., 70° C., 75° C., 80° C., or higher) provided that the elevated temperature does not irreversibly denature the albumin.

Following such a denaturation step albumin can be recovered in either the S3 supernatant or the P3 precipitate, depending upon the precipitant selected, the concentration of precipitant used, and/or the temperature at which the reaction is performed. The albumin recovered in S3 or P3 can have a purity of about 70%, 75%, 80%, 85%, 90%, 95%, 97%, 99%, or higher, and can be recovered at high yield (e.g., about 60%, 70%, 80%, 90%, 95%, 99%, or more relative to the albumin content of the S2 fraction). This partially purified albumin may be sufficiently pure for some purposes.

If higher purity is desired, the partially purified albumin can be further processed. Such further processing can include additional precipitation steps (as described above) and/or chromatography steps. Such chromatography steps preferably retain albumin in the flow-through fraction, which can greatly reduce the capacity or amount of chromatography media required and simplifies production at industrial scale (as described above). In some embodiments a single chromatography step can be employed. In other embodiments two or more chromatography steps can be used. In such embodiments a flow-through fraction containing albumin from a first chromatography step can be applied to a second chromatography step, from which a second flow-through fraction containing albumin is recovered. Suitable chromatography media include, but are not limited to ion exchange media, hydrophobic interaction media, mixed-mode media, affinity media, and pseudo-affinity media (e.g., dye media). The purified albumin recovered from such processing steps can have a purity of 80%, 85%, 90%, 95%, 97% 99%, or higher). In some embodiments no contaminating proteins are detectable upon SDS-PAGE of the purified albumin. The yield of purified albumin can be from 40%, 50%, 60%, 70%, 80%, 90%, 95%, 97%, 99%, or more relative to albumin content of the S2 fraction.

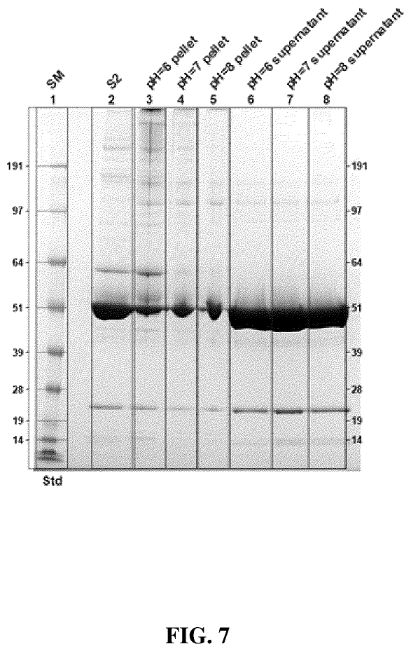

Inventors have found that treatment of S2 with caprylate at elevated temperature induces precipitation of the majority of non-albumin protein contaminants from S2. FIG. 7 shows results of treatment of S2 with app. 40 mM caprylate at different pH values, followed by incubation at 60° C. for one hour. As shown the majority of non-albumin contaminants are precipitated. Results from similar studies performed at 50° provide similar results. As shown, treatment with caprylate at pH 6 to 8 is effective in precipitating major contaminants from the S2 material while leaving albumin in solution.

Surprisingly, Inventors have found that albumin can be recovered at high purity (e.g., 90%, 95%, 97%, or higher) and high yield (e.g., 70%, 80%, 90%, or higher) from supernatants recovered from caprylate treatment as described above using ion exchange chromatography where albumin is recovered in flow-through from the ion exchange media. This advantageously avoids cumbersome bind, wash, and elute steps and dramatically reduces the volume of ion exchange media required. These features in turn support at-scale processes where 50 L to 5,000 L or more of plasma are processed.

In an example of such an albumin purification process, frozen plasma is thawed, and cryoprecipitate removed to generate a cryo-poor plasma. A precipitating salt (e.g., a citrate or acetate salt) is added to provide a final concentration of 10% to 13% or about 11% by weight, and the resulting mixture separated into supernatant (S1) and precipitate (P1) fractions. Albumin and IgG is retained in the S2 fraction, to which additional precipitating salt is added to give a concentration of from 22% to 30% by weight, or about 26% by weight. This generates a second precipitate (P2) and a second supernatant (S2). Most or all of the albumin is found in the second supernatant. This S2 can be processed for further purification of albumin, or it can be frozen for storage and thawed prior to processing for further purification of albumin.

The pH of S2 is adjusted to about 7 and sodium caprylate is added to give a final concentration of about 10 mM to 100 mM or about 40 mM caprylate. The resulting mixture is then incubated at about 60ºC for one hour and the resulting precipitate (P3) removed. The precipitate can be removed from the supernatant (S3) by any suitable method, including centrifugation, settling and decanting, and filtration. For example, following caprylate/heat treatment the resulting mixture can be passed through a depth filter, such as a PDD1™ depth filter (Cytiva) or a V100P™ filter (Cytiva). Surprisingly, Inventors have found that use of a PDD1™ depth filter is effective in reducing some low molecular weight (i.e., lower MW than albumin) contaminants that are not removed by the V100P™ depth filter.

S3 can be subjected to diafiltration in order to remove contaminants and/or provide suitable conditions for ion exchange chromatography. For example, S3 can be diafiltered using a 10 kD cutoff membrane or a 30 kD cutoff membrane. Buffers suitable for ion exchange typically have relatively low ionic strength (e.g., less than about 100 mM) and have a pH appropriate for the protein's isoelectric point and the nature of the fixed charge on the ion exchange media. For example, to recover albumin in a flow-through fraction the pH can be slightly acidic (e.g., pH 4.5 to pH 6.5, pH 5 to pH 6, or about pH 5.3) when a weak anion exchange chromatography media (e.g., DEAE media) is used. Surprisingly, when S3 was exchanged in to 50 mM acetate buffer at pH 5.3 and applied to a DEAE, approximately 97% pure albumin was recovered in the flow-through fraction as a second broad peak that followed an initial flow-through peak. This pattern suggests partial retention of albumin on the DEAE media. This degree of purity can be sufficient for pharmaceutical use. Accordingly, the albumin-containing peak obtained as a flow-through fraction from DEAE chromatography can be further processed for use, for example by nanofiltration and adjustment of protein concentration prior to filling.

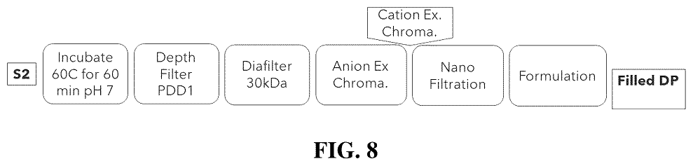

Optionally, a second chromatography step can be applied to this high purity flow-through peak to generate a polished albumin. For example, this albumin-containing flow-through peak can be applied to a small amount of cation exchange media (e.g., a cation exchange filter) to remove additional contaminants if required. Such a small amount of ion exchange media can be selected such that its capacity for albumin does not significantly impact yield (e.g., more than about 25%, 20%, 15% 10%, 5%, or less) while retaining tightly bound contaminants. A schematic representation of an albumin isolation method of the inventive concept is provided in FIG. 8.

As noted above, Inventors have also developed methods for the isolation of IgG from plasma using a combination of salt precipitation and ion exchange, with recovery of IgG at high yield and high purity from flow-through fractions from the ion exchange chromatography steps. These methods generate S1, P1, S2, and P2 fractions during salt precipitation steps that correspond to the S1, P1, S2, and P2 fractions described above for the isolation of albumin.

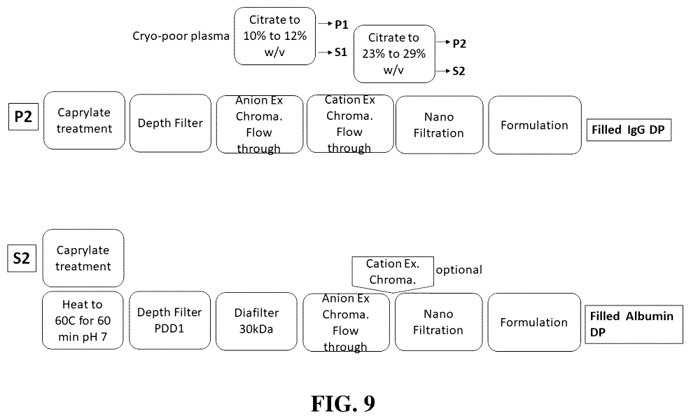

Embodiments of the inventive concept include methods in which IgG isolation and albumin isolation are combined to provide a single, integrated process for IgG and albumin isolation from plasma without the use of fractions obtained from binding and elution from chromatography media. In such methods, S2 is further processed for albumin isolation as described above for production of purified and polished albumin while P2 is further processed for IgG isolation using anion exchange and, in some embodiments, an additional cation exchange step where IgG is recovered from anion exchange and cation exchange steps in respective flow-through fractions. A schematic representation of such an integrated process is shown in FIG. 9.

Embodiments of the inventive concept include those in which additional proteins are recovered from S1, P1, S2, P2, S3, P3, and/or intermediate products of IgG and/or albumin isolation paths of the integrated process (e.g., by affinity chromatography). Examples of such additional proteins include alpha 1 antitrypsin.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method for reducing an environmental impact of industrial scale isolation of a first protein from a blood product, comprising:
(1) adding a precipitating salt to a volume of the blood product comprising the first protein, a second protein, a third protein, and a fourth protein at ambient temperature to generate a supernatant and a precipitate, wherein the blood product has a volume 2,000 L or more or is derived from 2,000 L or more of blood plasma;
(2) applying one of the supernatants or the precipitates to a first ion exchange media comprising a first charged group to generate a first flow-through fraction comprising the first protein and the third protein, wherein the second protein is retained on the first chromatography media; and
(3) applying the first flow-through fraction to a second ion exchange media comprising a second charged group to generate a second flow-through fraction comprising the first protein, wherein the first charged group and the second charged group have opposing charges, and wherein chromatography conditions are selected such that both the first protein and the third protein can bind to the second ion exchange media, and
wherein steps (1) to (3) are performed at ambient temperature and completed in less than 24 hours from initiation of step (1).

2. The method of claim 1, wherein the blood product is a product of a salt precipitation step applied to a blood plasma.

3. The method of claim 1, wherein the volume of the blood product is 5,000 L or more or is derived from 5,000 L of blood plasma or more.

4. The method of claim 1, wherein the precipitate is applied to the first chromatography media and the first protein is IgG.

5. The method of claim 1, wherein the environmental impact is energy consumption, wherein the blood product is cryo-poor plasma, wherein the first protein is IgG, wherein the precipitating salt is a citrate salt, and wherein the method provides an 89% reduction in energy requirements relative to an ethanol-precipitation method for isolating IgG from cryo-poor plasma at the volume.

6. The method of claim 1, comprising passing the supernatant or the precipitate through a filter media prior to application to the first chromatography media.

7. The method of claim 14, wherein the filter media is selected to selectively bind an activated clotting factor.

8. The method of claim 1, comprising capture and recycling of the precipitating salt.

9. The method of claim 1, wherein the method does not comprise recovery or reclamation of an organic solvent.

10. The method of claim 1, comprising recovery of the fourth protein from supernatant.

11. The method of claim 10, wherein the fourth protein is albumin.

12. The method of claim 11, comprising an additional step of adding caprylate to the supernatant to generate a caprylate treated supernatant and raising the temperature of the caprylate treated supernatant to about 40-70° C. to generate a third supernatant and a third precipitate, wherein the third supernatant comprises a crude albumin.

13. The method of claim 12, further comprising a step of adjusting pH of the third supernatant to from pH 4.5 to pH 7.0 and application to an anion exchange media to generate a third flow-through fraction comprising a purified albumin.

14. The method of claim 13, further comprising a step of applying the third flow through to a cation exchange media to generate a fourth flow-through comprising a polished albumin, wherein capacity of the cation exchange media is selected to bind less than 30% of albumin content of the first flow-through.
